(19) United States
(12) Patent Application Publication
(10) Pub. No.: US 2026/0134980 A1
(43) Pub. Date: May 14, 2026

(54) CLINICAL AI SAFETY, FAILURE MODE, AND CONTAINMENT GOVERNANCE SYSTEM

(71) Applicant: George William Bickerstaff, III, Greenwich, CT (US)

(72) Inventor: George William Bickerstaff, III, Greenwich, CT (US)

(21) Appl. No.: 19/445,482

(22) Filed: Jan. 10, 2026

(51) Int. Cl. G16H 40/20; G16H 15/00; G16H 40/67

(52) U.S. Cl. CPC G16H 40/20; G16H 40/67; G16H 15/00

(57) ABSTRACT

A clinical AI safety, failure mode, and containment governance system monitors AI-assisted clinical workflows for safety risks, detects failure modes, applies governed containment actions, and generates auditable safety records while preserving clinician authority and supporting regulatory-ready oversight.

### TECHNICAL FIELD

The present invention relates to clinical artificial intelligence safety and governance systems and, more particularly, to computer-implemented systems and methods for detecting, classifying, containing, and governing failure modes associated with artificial intelligence-assisted clinical workflows.

### BACKGROUND

Artificial intelligence systems integrated into clinical workflows may exhibit unexpected behavior, performance degradation, or emergent interactions when deployed in real-world healthcare environments.

Such failure modes may arise from data drift, integration errors, workflow misalignment, infrastructure instability, or unanticipated operational conditions.

Existing clinical AI deployments frequently lack real-time mechanisms for detecting unsafe conditions, limiting exposure, and documenting containment decisions in a governed and auditable manner.

Manual safety reviews and retrospective incident analysis are insufficient for managing AI-assisted workflows operating at clinical scale and speed.

Accordingly, there exists a need for a technical system that continuously monitors AI-assisted clinical workflows, detects failure modes, applies containment controls, and supports human-governed oversight without autonomously diagnosing medical conditions or prescribing treatment actions.

### SUMMARY OF THE INVENTION

The invention provides a computer-implemented clinical AI safety, failure mode, and containment governance system configured to monitor AI-assisted clinical workflows for predefined and emergent risk conditions.

The system detects safety anomalies, classifies failure modes, applies containment actions, and routes events through governed human review workflows.

The system preserves clinician authority by operating as a technical oversight and control-plane infrastructure rather than a clinical decision-making system.

All safety detections, containment actions, and governance decisions are recorded as immutable audit artifacts suitable for regulatory review and long-term oversight.

### Definitions (Alphabetical Order)

Audit Artifact refers to an immutable, time-stamped record capturing detection, classification, containment, and governance actions.
Containment Action refers to a system-initiated limitation applied to an AI-assisted workflow to reduce risk exposure.
Failure Mode refers to a detectable condition indicating degraded, unsafe, or unexpected AI system behavior.
Hazard Indicator refers to a measurable signal associated with elevated clinical or operational risk.
Monitoring Rule refers to a predefined condition used to evaluate workflow safety.
Risk Classification refers to a categorization of a detected failure mode by severity.
Safety Event refers to a logged occurrence associated with a detected failure mode.
Safety Governance Workflow refers to a structured, human-driven process for reviewing and resolving safety events.
System Degradation Refers to Performance Decline Relative to an established operational baseline.
Workflow Containment State Refers to a Restricted Operational state applied to an AI-assisted workflow.

### BRIEF DESCRIPTION OF THE DRAWINGS

FIG. 1 illustrates a clinical AI safety and containment system architecture.

FIG. 2 illustrates safety monitoring and hazard detection.

FIG. 3 illustrates failure mode classification and containment.

FIG. 4 illustrates governance review and corrective workflows.

FIG. 5 illustrates audit logging and regulatory readiness.

### DETAILED DESCRIPTION

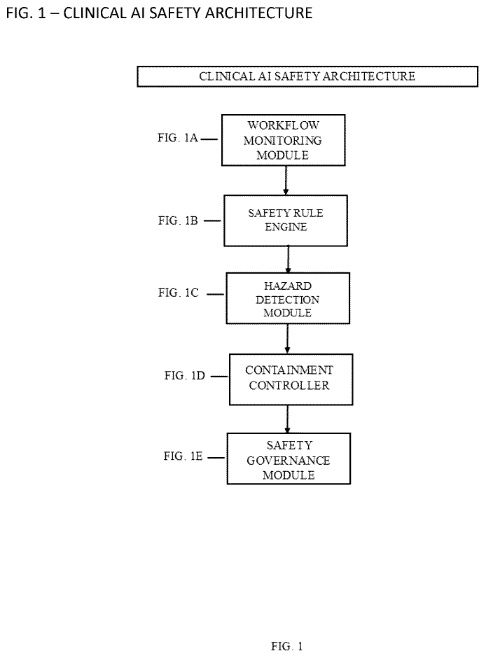

### FIG. 1—Clinical AI Safety Architecture

FIG. 1 illustrates a clinical AI safety governance system comprising workflow monitors, hazard detection engines, containment controllers, governance interfaces, and audit components. The system operates alongside AI-assisted clinical workflows without interfering with clinician decision authority. Deployment may occur within hospital infrastructure or secure clinical analytics platforms.

FIG. 1A—Workflow Monitoring Module. FIG. 1A Depicts a module configured to observe AI-assisted workflow execution characteristics. Observations include timing, frequency, and contextual attributes. Monitoring does not alter workflow execution.

FIG. 1B—SAFETY RULE ENGINE. FIG. 1B illustrates a safety rule evaluation engine applying monitoring rules to observed workflow data. Evaluation is deterministic and configurable. Results are auditable.

FIG. 1C—Hazard Detection Module. FIG. 1C Depicts a Module identifying deviations from expected operational behavior. Detected hazards are logged as safety events. Detection is automated.

FIG. 1D—CONTAINMENT CONTROLLER. FIG. 1D illustrates a controller applying containment actions in response to detected failure modes. Actions are reversible and governed. Clinical authority is preserved.

FIG. 1E—SAFETY GOVERNANCE MODULE. FIG. 1E depicts a module recording safety events and containment actions. Records are immutable and time-stamped. Stored artifacts support oversight.

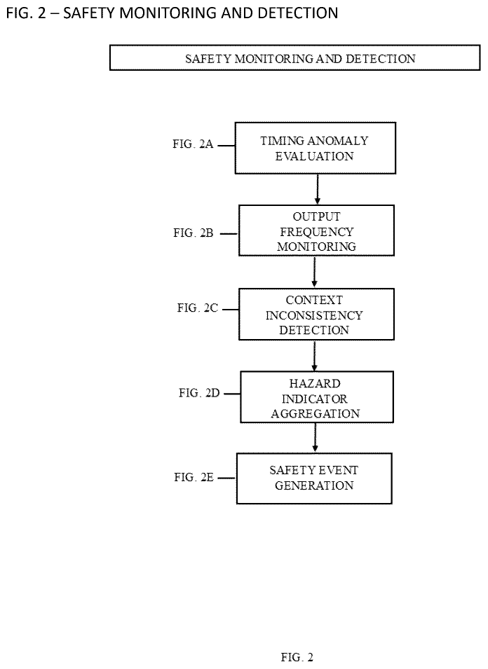

### FIG. 2—Safety Monitoring and Detection

FIG. 2 illustrates continuous monitoring of AI-assisted workflows for safety risks. Monitoring is rule-based and configurable. Monitoring does not generate clinical judgments.

FIG. 2a—Timing Anomaly Evaluation. FIG. 2A depicts

evaluation of workflow execution timing. Timing anomalies may indicate degradation. Anomalies are recorded.

FIG. 2B—OUTPUT FREQUENCY MONITORING. FIG. 2B illustrates monitoring of output frequency and volume. Unexpected changes may indicate failure modes. Observations are logged.

FIG. 2C—CONTEXT INCONSISTENCY DETECTION. FIG. 2C depicts detection of contextual inconsistencies across workflow inputs. Inconsistencies may reflect integration issues. Detection is automated.

FIG. 2D—HAZARD INDICATOR AGGREGATION. FIG. 2D illustrates aggregation of hazard indicators across signals. Aggregation supports risk assessment. Traceability is preserved.

FIG. 2E—SAFETY EVENT GENERATION. FIG. 2E depicts generation of safety event records. Records capture detected hazards and metadata. Records are versioned.

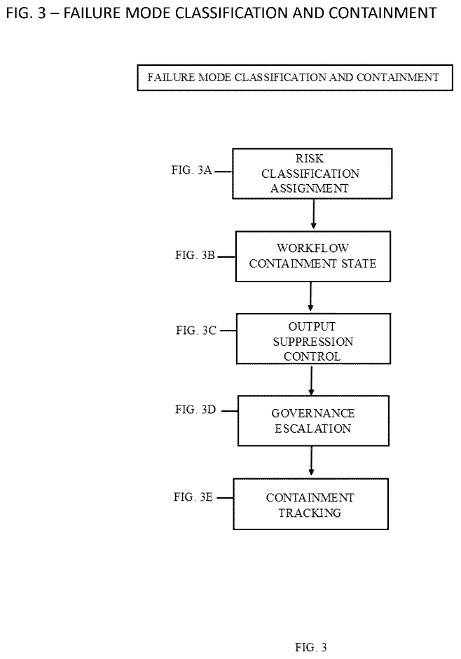

### FIG. 3—Failure Mode Classification and Containment

FIG. 3 illustrates classification of detected failure modes and application of containment actions. Classification informs response selection. Responses are governed.

FIG. 3A—RISK CLASSIFICATION ASSIGNMENT. FIG. 3A depicts assignment of severity-based risk classifications. Thresholds are configurable. Classification outcomes are logged.

FIG. 3B—WORKFLOW CONTAINMENT STATE. FIG. 3B illustrates transition of workflows into containment states. Containment limits exposure. Clinical decisions remain unaffected.

FIG. 3C—OUTPUT SUPPRESSION CONTROL. FIG. 3C depicts selective suppression of AI-assisted outputs. Suppression reduces risk propagation. Suppression is auditable.

FIG. 3D—GOVERNANCE ESCALATION. FIG. 3D illustrates escalation of safety events to governance stakeholders. Escalation ensures awareness. Escalation does not mandate actions.

FIG. 3E—CONTAINMENT TRACKING. FIG. 3E depicts tracking of containment duration and scope. Tracking supports review. Tracking is immutable.

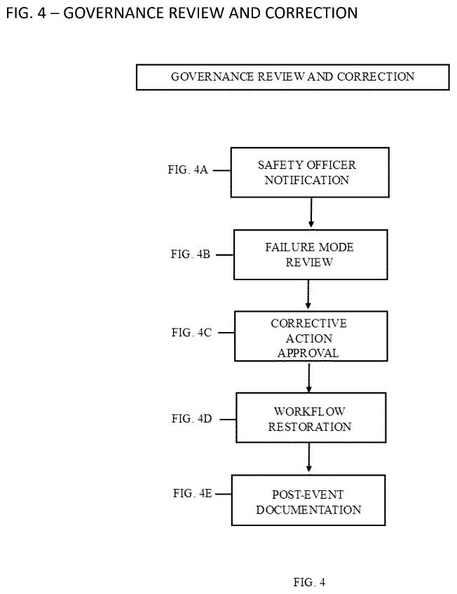

### FIG. 4—Governance Review and Correction

FIG. 4 illustrates governance workflows for reviewing and resolving safety events. Workflows are human-driven. Outcomes are documented.

FIG. 4A—SAFETY OFFICER NOTIFICATION. FIG. 4A depicts notification of safety officers and clinical leadership. Notifications are informational. Notifications are logged.

FIG. 4B—FAILURE MODE REVIEW. FIG. 4B illustrates structured review of detected failure modes. Reviews require authorization. Review outcomes are recorded.

FIG. 4C—CORRECTIVE ACTION APPROVAL. FIG. 4C depicts approval of corrective actions. Actions may include configuration changes. Approval is documented.

FIG. 4D—WORKFLOW RESTORATION. FIG. 4D illustrates restoration of workflows from containment states. Restoration requires human approval. Events are logged.

FIG. 4E—POST-EVENT DOCUMENTATION. FIG. 4E depicts post-event analysis and documentation. Analysis supports learning. Documentation is archived.

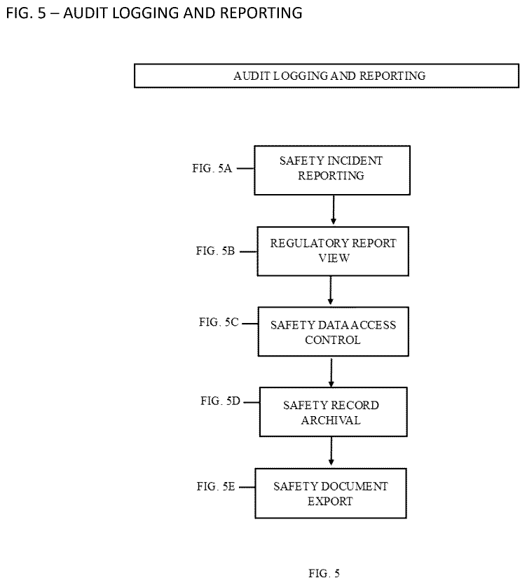

### FIG. 5—Audit Logging and Reporting

FIG. 5 illustrates safety audit logging and reporting. Logs support internal and external review. Reporting is standardized.

FIG. 5A—SAFETY INCIDENT REPORTING. FIG. 5A depicts generation of safety incident reports. Reports summarize events and responses. Reports are versioned.

FIG. 5B—REGULATORY REPORT VIEW. FIG. 5B illustrates regulator-aligned safety reporting views. Views support audits. Views are controlled.

FIG. 5C—SAFETY DATA ACCESS CONTROL. FIG. 5C depicts role-based access to safety records. Access is logged. Controls are enforced.

FIG. 5D—SAFETY RECORD ARCHIVAL. FIG. 5D illustrates archival of safety governance records. Records are retained per policy. Oversight is supported.

FIG. 5E—SAFETY DOCUMENT EXPORT. FIG. 5E depicts export of safety documentation. Export formats are standardized. Patient identifiers are excluded.

### Illustrative Operational Example

In one example, an AI-assisted triage system exhibits a sudden increase in alert frequency. The monitoring module detects the anomaly and classifies it as a moderate-risk failure mode.

A containment action suppresses non-critical alerts while escalating the event to governance stakeholders. Clinicians retain full authority over care decisions.

Following review, the workflow is restored and documentation is archived. The system does not autonomously diagnose or prescribe treatment.

1. A computer-implemented clinical AI safety governance system comprising one or more processors and memory storing instructions that cause the system to monitor AI-assisted clinical workflows, detect failure modes, classify risk, apply containment actions, and generate immutable audit artifacts, wherein the system does not autonomously diagnose a medical condition or prescribe treatment.

2. A method comprising monitoring AI-assisted clinical workflows, detecting hazard indicators, classifying failure modes, initiating containment actions, and recording governance decisions.

3. A non-transitory computer-readable medium storing instructions that cause one or more processors to perform the method of claim 2.
DEPENDENT CLAIMS

4. The system of claim 1, wherein containment actions include workflow suppression.

5. The system of claim 1, wherein risk classifications are severity-based.

6. The system of claim 1, wherein restoration requires human approval.

7. The system of claim 1, wherein safety events are immutable once logged.

8. The system of claim 1, wherein hazard indicators include timing anomalies.

9. The method of claim 2, wherein post-event analysis is documented.

10. The system of claim 1, wherein regulator-aligned reports are generated.
